SEQUENCE LISTING (14 sequences)

SEQ ID NO: 1 - protein, 473 aa, Homo sapiens
MAQHDFAPAWLNFPTPPSSTKSSLNFEKHSENFAWTENRYDVNRRRHNSSDGFDSAIGRPNGGNFGRKEKNGWRTHGRNGTENINHRGGYHGGSSRSRSSIFHAGKSQGLHENNIPDNETGRKEDKRERKQFEAEDFPSLNPEYEREPNHNKSLAAGVWEYPPNPKSRAPRMLVIKKGNTKDLQLSGFPVVGNLPSQPVKNGTGPSVYKGLVPKPAAPPTKPTQWKSQTKENKVGTSFPHESTFGVGNFNAFKSTAKNFSPSTNSVKECNRSNSSSPVDKLNQQPRLTKLTRMRTDKKSEFLKALKRDRVEEEHEDESRAGSEKDDDSFNLHNSNSTHQERDINRNFDENEIPQENGNASVISQQIIRSSTFPQTDVLSSSLEAEHRLLKEMGWQEDSENDETCAPLTEDEMREFQVISEQLQKNGLRKNGILKNGLICDFKFGPWKNSTFKPTTENDDTETSSSDTSDDDDV

SEQ ID NO: 2 - DNA, 3835 bp, Homo sapiens
agaacattgcggatcgggtcggcgccattttgggactgagactggttgtgggggagggaaaagcggcaaaaggggattattcaaagtaccgaaaaccttctcccgggatcaggcgcggcggcacccccaggccaggggcacctctggtggggcagaaggtgattgaattactcagatatgaagatcatcatctaggttttgtgtaaaaggccctggatattttaagtggccattttggatttacagtgtttttggataattttgccccagaagtttattaaaattggcaagaatcgtctgtgaagtgaattgatagtagtgaacaattcagcaagctacttaaaaagagacccaggcagcatttcttcagtattttggttcaaacggattatataactggttacagtatttcagctggtggtaatttttgcctccccttcccccaccccgttgttggggttcttcagccgaaactgagagacgttgatttgtgtactgagtagtttcagcagtttcaaatgactgagtattgctgaagtttcatggcagtttatttttacctttattgaaagttttaggaatttttgacttcagctctttcatgtcacaatgggacactttttctgaatgaagagattgaaagaatacagagtttttttccttttatcttttatttacgtggaaatttaagatgttgcagttttccggcagcatggtagtattgagatagctatgtgtgtctctgtatatgctgatgtttaggaatgctcttcagatgtgaaattttctttttgtttttgctttttggctcgtaaattggatatttcatctggagtggacaagtacaacagtggcaagtacatggaataataaagaagactttgatcttaaatctaaagaacttggctaattcgggagatagccatatgaaaactttaaaacagaagtatgggtagctgacttgaagtaactctatgtcaaatagtcgtaggttaagtatcttcaaagaacttcgatattatttcagaggatacaaaataaaaatacaaactggaaaataaagattacagagaaaaaaccaacaccttcctgtgcagtcctgttggaatttggacttgccatgaggtgttgaagccttgtttcactgagttggagagactggacctaaatggcgcagcatgactttgctccagcctggcttaatttccctactccaccatcatcaacaaagtcgtcattgaattttgagaagcattctgaaaactttgcatggacagagaatcgttatgatgtgaaccgtcgacgacacaactcttcagatggctttgattctgctattggacgtcctaatggaggtaactttggaaggaaagaaaaaaatggatggcgtacacatggaagaaatggtacagaaaacataaatcatcgaggtggataccatggtggaagttcccgttctcgtagcagtattttccatgcaggaaaaagccaaggactacatgaaaacaacatacctgacaatgaaaccgggaggaaagaagacaagagagaacgcaaacagtttgaagctgaggattttccgtctttaaatcctgagtatgagagagaaccaaatcacaataagtctttagctgcaggtgtgtgggaatatcctccgaatcctaaatctagagctccaaggatgctggtcattaagaaaggtaatacaaaagacttacagctatctggattcccagtagtaggaaatcttccgtcacagccagttaagaatggaactggtccaagtgtttataaaggtttagtccctaaacctgctgctccacctacaaaacctacacaatggaaaagccaaacaaaagaaaataaagttggaacttctttccctcatgagtccacatttggcgttggcaactttaatgcttttaaatcaactgccaagaactttagtccatctacaaattcagtgaaagagtgtaatcgctcaaattcctcttctcctgttgacaaacttaatcagcagcctcgtctaaccaaactgacacgaatgcgcactgataagaagagtgaatttttgaaagcattgaaaagagacagagtagaagaggaacatgaagatgaaagccgtgctggctcagagaaggatgacgactcatttaatttacataacagcaatagtactcaccaagaaagggatataaaccgaaacttcgatgaaaatgaaattcctcaagagaatggcaatgcctcagtgatttcccagcagatcattcggtcttcaaccttcccacaaactgatgttctttcaagttcacttgaggcagaacacagattgttaaaggaaatgggctggcaggaagacagtgaaaatgatgaaacatgtgctcccttaactgaggatgaaatgagagaattccaagttattagtgaacagttacagaagaatggtctgagaaaaaatggtattttgaaaaatggcttgatctgtgacttcaagtttggaccgtggaagaacagcactttcaaacccacaactgagaatgatgacacagagacaagtagcagtgatacatcagatgacgacgatgtgtgaaggatttcctaacagctttagaaatcttagtgtgatacatctctcatacagtttggggtgaattgtaaaaatgaagaactataatttatgtagtgaaataccccattagaagaggattttttgggggacttcaatatgaagaaaaccaagaatgttttgttgggctgtgttgaacattatttctttgtaaatgaatgttgtaggaatgaggacttgggttggtccaacattgactttcttcatcactgcaacatttctctgactagcaatgtgacgatgtaacaaatgagattttctcatttaataataaaaaattgtgtaatgttttgcaaagcttctgtcttaaaatgtccaggtcttaagaaaaaaggcagcttacactgttttgcttgcagagtcatatctttttcgtacaatggaaatcctcaagtccactttgtgcggtctccctctccttcccccaaaaaacaacaacaacaaaacaaaaaccaaaaaggaaaatgtagcatgttggctaaaactggagcaaagtgcactaaaacaatttcctgaactcacctgttgtactattcaccttttaaaccataaattgctctttagccatttgtagtgcagtaaatgttacaggaaaagacttggcacattttcttccaaattttaagaggtgattttcaaaagctttattggggtatgttgtcagaccagggttttcagagttgatggaaaagagtcttgtgagaaaacttattttgataaattattacacacgcagaaaaactgatcacactgactggatctgtccacgacatggaaaataaactggattttcagaatattgttgttttctgtagtgttcaaggtattgtttctaaacataaacatactctaaacatgctttattcacttgttaaagtcatacttttaaaagtaataccttactaaagatggtgattacttttccgaggtcagaaaaggaaagctaagcgttttcattatcaaatacacaagcttattaaatgaatgactgttaactactttattttcatttgcacattaattttggaattgtttctgttttgctgctgacggaaatactattttggctctgtgtatatttgtattttgatttttctggtttgtttacccccatttgcttttagctcccccttatgtttaaatatattctaacttatgtaaagagcataatcttagagcaaaaatacttgaggttttatgtcagatcta

SEQ ID NO: 3 - protein, 946 aa, Homo sapiens
MVPWVRTMGQKLKQRLRLDVGREICRQYPLFCFLLLCLSAASLLLNRYIHILMIFWSFVAGVVTFYCSLGPDSLLPNIFFTIKYKPKQLGLQELFPQGHSCAVCGKVKCKRHRPSLLLENYQPWLDLKISSKVDASLSEVLELVLENFVYPWYRDVTDDESFVDELRITLRFFASVLIRRIHKVDIPSIITKKLLKAAMKHIEVIVKARQKVKNTEFLQQAALEEYGPELHVALRSRRDELHYLRKLTELLFPYILPPKATDRRSLTLLIREILSGSVFLPSLDFLADPDTVNHLLIIFIDDSPPEKATEPASPLVPFLQKFAEPRNKKPSVLKLELKQIREQQDLLFRFMNFLKQEGAVHVLQFCLTVEEFNDRILRPELSNDEMLSLHEELQKIYKTYCLDESIDKIRFDPFIVEEIQRIAEGPHIDVVKLQTMRCLFEAYEHVLSLLENVFTPMFCHSDEYFRQLLRGAESPTRNSKLNRGSLSLDDFRNTQKRGESFGISRIGSKIKGVFRSTTMEGAMLPNYGVAEGEDDFIEEGIVVMEDDSPVEAVSTPNTPRNLAAWKISIPYVDFFEDPSSERKEKKERIPVFCIDVERNDRRAVGHEPEHWSVYRRYLEFYVLESKLTEFHGAFPDAQLPSKRIIGPKNYEFLKSKREEFQEYLQKLLQHPELSNSQLLADFLSPNGGETQFLDKILPDVNLGKIIKSVPGKLMKEKGQHLEPFIMNFINSCESPKPKPSRPELTILSPTSENNKKLFNDLFKNNANRAENTERKQNQNYFMEVMTVEGVYDYLMYVGRVVFQIPDWLHHLLMGTRILFKNTLEMYTDYYLQCKLEQLFQEHRLVSLITLLRDAIFCENTEPRSLQDKQKGAKQTFEEMMNYIPDLLVKCIGEETKYESIRLLFDGLQQPVLNKQLTYVLLDIVIQELFPELNKVQKEVTSVTSWM

SEQ ID NO: 4 - DNA, 3145 bp, Homo sapiens
aaaaaactgccggtaagcgtctgtgtgcgccgccaagtcggtggggcggggacgcgaggtgtggatggggggtcgccttgacctctgcctcagccagtagcgcagcctcggcctcgccgttacggagatggtgccctgggtgcggacgatggggcagaagctgaagcagcggctgcgactggacgtgggacgcgagatctgccgccagtacccgctgttctgcttcctgctgctctgtctcagcgccgcctccctgcttcttaacaggtatattcatattttaatgatcttctggtcatttgttgctggagttgtcacattctactgctcactaggacctgattctctcttaccaaatatattcttcacaataaaatacaaacccaagcagttaggacttcaggaattatttcctcaaggtcatagctgtgctgtttgtggtaaagtgaaatgtaaacgacataggccttctttgctacttgaaaactaccagccatggctagacctgaaaatttcttccaaggttgatgcatctctctcagaggttcttgaattagtgttggaaaactttgtttatccgtggtacagggatgtgacagatgatgaatcctttgttgatgaactgagaataacattacgtttttttgcatctgtcttaataagaaggattcacaaggtggatattccatctattataaccaagaaactattaaaagcagcaatgaagcatatagaagtgatagttaaagccagacagaaagtaaaaaatacagagtttttacagcaagctgctttagaagaatatggtccagagcttcatgttgctttgagaagtcgaagagatgaattgcactatttaaggaaacttactgaactgctttttccttatattttgcctcctaaagcaacagaccgcagatctctgaccttacttataagagagattctgtctggctctgtgttccttccttctttggatttcctagctgatccagatactgtgaatcatttgcttatcatcttcatagatgacagtccacctgaaaaagcaactgaaccggcttctcctttggttccattcttgcagaaatttgcagaacctagaaataaaaagccatctgtgctgaagttagaattgaagcaaatcagagagcaacaagatcttttatttcgttttatgaactttctgaaacaagaaggcgcagtgcacgtgttgcagttttgtttgactgtggaggaatttaatgatagaattttacgaccagaattatcaaatgatgaaatgctgtctcttcatgaagaattgcagaagatttataaaacatactgtttggatgaaagtattgacaaaattagatttgatcccttcattgtagaagagattcaaagaattgctgaaggcccacacatagatgttgtgaaacttcaaactatgagatgtctttttgaagcatatgaacatgttctttcccttttggagaatgtatttactcctatgttctgccatagtgatgagtatttcagacaacttttaagaggtgcagaatcaccaacacgcaattcaaaattgaacagaggtagcctaagtttggatgattttcggaacacacagaaaaggggagaatcatttggaatcagcagaataggtagcaaaattaaaggagtattcagaagtaccacaatggagggagctatgttgcctaattatggtgtagctgaaggtgaagatgattttattgaagaaggtattgttgtaatggaagatgattctccagtggaggctgtgagcacacctaatactccccgaaaccttgctgcatggaaaattagcattccatatgtagacttttttgaggatccctcctctgaaaggaaggagaaaaaagaaagaattcctgtgttttgtattgatgttgaaagaaatgatagaagagcagttggacacgagcctgaacattggtctgtctatagaagatatcttgaattctatgtacttgaatcaaaactaacagaatttcatggtgcatttcctgatgcccagcttccttctaagaggatcattggccccaaaaattatgaattcttaaagtcaaagagggaagagttccaagaatatctacagaaacttctgcagcatccagaactgagtaatagtcaacttctggcagactttctttcccctaatggtggggaaacacaatttcttgataagatactaccagatgtaaatcttgggaaaattataaaatctgttcctggaaaactaatgaaagagaaaggtcagcatttggaaccttttatcatgaatttcattaattcttgtgagtctccaaagcctaaaccaagtagaccagaactgaccattctcagccctacttcagaaaacaacaagaagcttttcaatgatctgtttaaaaataatgcaaaccgtgctgaaaatacagagagaaagcaaaatcagaattattttatggaggtgatgactgtagaaggagtctatgattacctgatgtatgtaggacgggtagttttccagattcctgactggcttcatcatctcttaatgggaactcgaatcctctttaaaaacaccctggaaatgtatactgattactatcttcagtgtaaactagaacagctatttcaggagcaccgtttggtctcactcataacacttctcagagatgctatattctgtgaaaacactgaacctcgctctctccaagataagcaaaaaggagcaaaacagacttttgaagaaatgatgaattacattccagatctgttagtcaagtgtattggtgaggaaaccaagtatgaaagcatcagacttctgtttgatggcttacagcaaccagtactcaacaagcagctgacttatgttttattggacattgtgatacaggaactgtttccagagctcaataaggtacaaaaggaagttacctctgtgacatcttggatgtaaacacttggatttggtatagaataacccattgaaatttctgctgtgcgagggtggtagaaatttacttttttgggtatattctatatatattatgtacatcgctgtctgaaattttagttattttttgtttttaataaagactaacacaacttaatgattaaaaaaaaaaaaaaaaaa

SEQ ID NO: 5 - protein, 950 aa, Homo sapiens
MTWRMGPRFTMLLAMWLVCGSEPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 6 - DNA, 4117 bp, Homo sapiens
taagttaccccgatgacttggtttggaaggggttaaggcaccagtcatcctcttctaaagtgatttatgatgatgtgtggagtttaaaaactttaccccaccccaaagaacagccctctcactcctcactgagtccactctgaacgtgctaaaatgggaaggaggcggtgttttgatgatctgttaaattcttagtgaagtttccttgatttccagtggctgctgttgtttgagtttggtttggagcaaaactgaggtagtcctaacatttctgggactgaatccaggcaagagaaagaagaaaaagaagaagaaaaagaggaggaaaaaggtagggagaaataaagggaggagagaagcacagtgaaaaaaaaaaaaagtcccttttcgacatcacattcctgtgttttccctcagcctggaaaacatatgaatcccagtgcttttacgcccggaaacaaagagactaagccagactatgggggaaagggagataagaaggatcctggaactttaaagagggaaagagtgagattcagaaatcgccaggactggactttaagggacgtcctgtgtcagcacaagggactggcacacacagacacacgagaccgaggagaaactgcagacaaatggagatacaaagacttagaaggacagctcctttcacctcatcctacttgtccagaaggtaaaaagacacagccagaaagaaaaggcatcggctcagctctcagatcaggacaggctgtggatctgtggcggtactctgaaagctggagctgcagcacaccccttttgtattgctcaccctcggtaaagagagagagggctgggaggaaaagtagttcatctaggaaactgtcctgggaaccaaacttctgatttcttttgcaaccctctgcattccatctctatgagccaccattggattacacaatgacatggagaatgggaccccgtttcactatgctgttggccatgtggctagtgtgtggatcagaaccccacccccatgccactattagaggcagccacggaggacggaaagtgcctttggtttctccggacagcagtaggccagctcggtttctgaggcacactgggaggtctcgcggaattgagagatccactctggaggaaccaaaccttcagcctctccagagaaggaggagtgtgcccgtgttgagactagctcgcccaacagagccgccagcccgctcggacatcaatggggccgccgtgagacctgagcaaagaccagcagccaggggctctccgcgtgagatgatcagagatgaggggtcctcagctcggtcaagaatgttgcgtttcccttcggggtccagctctcccaacatccttgccagctttgcagggaagaacagagtatgggtcatctcagcccctcatgcctcggaaggctactaccgcctcatgatgagcctgctgaaggacgatgtgtactgtgagctggcggagaggcacatccaacagattgtgctcttccaccaggcaggtgaggaaggaggcaaggtgagaaggatcaccagcgagggccagatcctggagcagcccctggaccctagcctcatccctaagctgatgagcttcctgaagctggagaagggcaagtttggcatggtgctgctgaagaagacgctgcaggtggaggagcgctatccatatcccgttaggctggaagccatgtacgaggtcatcgaccaaggccccatccgtaggatcgagaagatcaggcagaagggctttgtccagaaatgtaaggcctctggtgtagagggccaggtggtggcggaggggaatgacggtggagggggagcaggaaggccaagcctgggcagcgagaagaagaaagaggacccaaggagagcacaagtcccaccaaccagagagagtcgggtgaaggtcctgagaaaactggccgccactgcaccagctttgccccaacctccctcaacccccagagccaccacccttcctcctgccccagccacaacagtgactcggtccacgtcccgggcggtaacagttgctgcaagacctatgaccaccactgcctttcccaccacgcagaggccctggaccccctcaccctcccacaggccccctacaaccactgaggtgatcactgccaggagaccctcagtttcagagaatctttaccctccatcccggaaggatcagcacagggagaggccacagacaaccaggaggcccagcaaggccaccagcttggagagcttcacaaatgcccctcccaccaccatctcagaacccagcacaagggctgctggcccaggccgtttccgggacaaccgcatggacaggcgggaacatggccaccgagacccaaatgtggtgccaggtcctcccaagccagcaaaggagaaacctcccaaaaagaaggcccaggacaaaattcttagtaatgagtatgaggagaagtatgacctcagccggcctactgcctctcagctggaggacgagctgcaggtggggaatgttccccttaaaaaagcaaaggagtctaaaaagcatgaaaagcttgagaaaccagagaaggagaagaaaaaaaagatgaagaatgagaacgcagacaagttacttaagagtgaaaagcaaatgaagaagtctgagaaaaagagcaagcaagagaaagagaagagcaagaagaaaaaaggaggtaaaacagaacaggatggctatcagaaacccaccaacaaacacttcacgcagagtcccaagaagtcagtggccgacctgctggggtcctttgaaggcaaacgaagactccttctgatcactgctcccaaggctgagaacaatatgtatgtgcaacaacgtgatgaatatctggaaagtttctgcaagatggctaccaggaaaatctctgtgatcaccatcttcggccctgtcaacaacagcaccatgaaaatcgaccactttcagctagataatgagaagcccatgcgagtggtggatgatgaagacttggtagaccagcgtctcatcagcgagctgaggaaagagtacggaatgacctacaatgacttcttcatggtgctaacagatgtggatctgagagtcaagcaatactatgaggtaccaataacaatgaagtctgtgtttgatctgatcgatactttccagtcccgaatcaaagatatggagaagcagaagaaggagggcattgtttgcaaagaggacaaaaagcagtccctggagaacttcctatccaggttccggtggaggaggaggttgctggtgatctctgctcctaacgatgaagactgggcctattcacagcagctctctgccctcagtggtcaggcgtgcaattttggtctgcgccacataaccattctgaagcttttaggcgttggagaggaagttgggggagtgttagaactgttcccaattaatgggagctctgttgttgagcgagaagacgtaccagcccatttggtgaaagacattcgtaactattttcaagtgagcccggagtacttctccatgcttctagtcggaaaagacggaaatgtcaaatcctggtatccttccccaatgtggtccatggtgattgtgtacgatttaattgattcgatgcaacttcggagacaggaaatggcgattcagcagtcactggggatgcgctgcccagaagatgagtatgcaggctatggttaccatagttaccaccaaggataccaggatggttaccaggatgactaccgtcatcatgagagttatcaccatggatacccttactgagcagaaatatgtaaccttagactcagccagtttcctctgcagctgctaaaactacatgtggccagctccattcttccacactgcgtactacatttcctgcctttttctttcagtgtttttctaagactaaataaatagcaaactttcacctattcatgagttattattgaaacctcaaatcataaagacatttaaaagaattgtttttctaactggaggggctctagtgctaaataatagtactgaaaattgatattattttccttttcttatatgaaggaccttatttggcatataaaattttataaaatatgtaaaaaaaaaa

SEQ ID NO: 7 - DNA, 20 bp, Artificial Sequence
  Description of Artificial Sequence Primer
taatacgactcactataggg

SEQ ID NO: 8 - DNA, 17 bp, Artificial Sequence
  Description of Artificial Sequence Primer
atttaggtgacactata

SEQ ID NO: 9 - DNA, 31 bp, Artificial Sequence
  Description of Artificial Sequence Primer
tttcacacaggaaacaggaaacagctatgac

SEQ ID NO: 10 - DNA, 24 bp, Artificial Sequence
  Description of Artificial Sequence Primer
cgccagggttttcccagtcacgac

SEQ ID NO: 11 - DNA, 20 bp, Artificial Sequence
  Description of Artificial Sequence Primer
ggctaattcgggagatagcc

SEQ ID NO: 12 - DNA, 20 bp, Artificial Sequence
  Description of Artificial Sequence Primer
caacacctcatggcaagtcc

SEQ ID NO: 13 - DNA, 28 bp, Artificial sequence
  Description of Artificial Sequence Primer
cctaaatctagagcgtcgacgatgctgg

SEQ ID NO: 14 - DNA, 23 bp, Artificial sequence
  Description of Artificial Sequence Primer
aagctgttagtcgacccttcaca

(19) United States
(12) Patent Application Publication
(10) Pub. No.: US 2004/0132035 A1
(43) Pub. Date: Jul. 8, 2004

(54) Markers of unstable atherosclerotic plaques

(76) Inventors: Matthias Joseph A P Daemen, Maastricht (NL); Catharina Barbara J M Cleutjens, Susteren (NL); Guido Jenny R Zaman, Oss (NL)

Correspondence Address: HOFFMANN & BARON, LLP, 6900 JERICHO TURNPIKE, SYOSSET, NY, 11791, US

(21) Appl. No.: 10/467,369

(22) PCT Filed: Feb. 5, 2002
(86) PCT No.: PCT/EP02/01327

(30) Foreign Application Priority Data
Feb. 7, 2001 (EP) 01200439.6

(51) Int. Cl. C12Q 1/68; G01N 33/53; C07H 21/04

(52) U.S. Cl. 435/6; 435/7.1; 536/23.1

(57) ABSTRACT

The present invention relates to (the use of) polynucleotides differentially expressed in ruptured and stable atherosclerotic plaques as marker for atherosclerosis, prevention and treatment of atherosclerosis disorders.

The invention relates to the use of polynucleotides differentially expressed in ruptured and stable atherosclerotic plaques as marker for atherosclerosis, a method for determining the presence of said polynucleotides in a sample, a method for determining the presence of amino acids encoded by said polynucleotides in a sample, a diagnostic process wherein the expression level of said polynucleotides is determined, a diagnostic process wherein a sample is analyzed for the presence of said amino acid sequences as well as to a newly identified polynucleotide and the amino acid sequence encoded by that polynucleotide.

Atherosclerosis is a major problem in the western world and is the main cause of cardiovascular disease and deaths. It is a systemic chronic progressive disease affecting all major arteries. Atherosclerotic cardiovascular disease comprises a number of pathological conditions, such as acute coronary syndromes like ischemic (or coronary) heart disease (MID), stroke, and peripheral vascular disease.

Although it may take at least 30 to 40 years to become clinically manifest, one may conclude that atherosclerosis—though not always in severe forms—affects all adult individuals in the western world.

Endothelial dysfunction is one of the initiating events of chronic atherosclerosis, a slowly growing atherosclerotic plaque that encroaches the lumen and reduces the lumen. Endothelial dysfunction is associated with an apparent decrease in the synthesis of the vasodilator nitric oxide (NO). The subsequent development of the atherosclerotic lesion progresses through five stages, from early lesion to stenotic or thrombogenic and occlusive plaque. The different plaque types are defined by histological criteria (Stary, et al., Circulation 1999; 92: 1355-1374). Most clinical atherosclerotic symptoms (about two-thirds of coronary occlusions) are related to the transition of a stable atherosclerotic plaque into a ruptured atherosclerotic plaque. Rupture of unstable atherosclerotic plaques is characterised by a sudden activation of the clotting system, leading to a sudden occlusion of the lumen (thrombosis) (Libby, Circulation 1995; 91: 2844-2850; Dollery, et al., Circ. Res. 1995; 77: 863-868; Davies, Circulation 1996; 94: 2013-2020). Treatments that increase or maintain plaque stability may therefore for instance decrease the risk of coronary syndromes in patients with IHD, or decrease the risk of other clinical events associated with cardiovascular disease.

Patient groups in which atherosclerosis is the major cause of disease include patients with a myocardial infarction, angina pectoris, unstable angina, cerebral ischemia and infarction, dementia, and peripheral and intestinal ischemia. Patients at high risk for developing (premature) symptoms of atherosclerosis are those that have high serum cholesterol levels (in low density lipoprotein (LDL) or very low density lipoprotein (VLDL) particles), or high levels of triglycerides, lipoprotein (a), or fibrinogen, or those people that smoke, have hypertension, have diabete mellitus, or have familial (genetic) disorders in their lipoprotein metabolism, such as familial combined hyperlipidemia. All these patients may benefit from the utility of unstable plaque specific diagnostics/therapeutics.

Although it is known that a ruptured plaque causes the majority of clinical symptoms of atherosclerotic cardiovascular disease (Ross R., N Engl J Med 1999; 340:115-26; Libby P., J Intern Med 2000; 247:349-58; Zaman A. G., et al., Atherosclerosis 2000; 149:251-66.), it is not unravelled yet which factors and molecular mechanisms are responsible for the transition of a stable plaque into a ruptured plaque.

The morphology of ruptured plaques is well described (Stary H. C., et al., Arterioscler Thromb Vasc Biol 1995; 15:1512-31; Virmani R, et al., Arterioscler Thromb Vasc Biol 2000;20:1262-75), however, specific markers to identify ruptured plaques or plaques prone to rupture in vivo are not available (Kullo I. J., et al., Ann Intern Med 1998; 129:1050-60).

In an attempt to shed more light on the possible molecular mechanisms involved in the onset and progression of atherosclerosis, several studies compared gene expression of activated human umbilical vein endothelial cells and vascular smooth muscle cells to non-activated cells (Lu K. P., et al., Biochem Biophys Res Commun 1998; 253:828-33; Sato N., et al., J Biochem (Tokyo) 1998; 123:1119-26; De Waard V, et al., Gene 1999; 226:1-8; Horrevoets A. J., et al., Blood 1999; 93:3418-3431; De Vries C. J., et al., J Biol Chem 2000: 275:31:23939-47). These studies in cell lines, revealed differential regulation of genes involved in leukocyte trafficking, cell cycle control and apoptosis. However, although cell lines do provide a reproducible source of RNA, it remains to be determined whether gene expression in vitro mimics gene expression in vivo. Others (Hiltunen M. O., Curr Opin Lipidol 1999; 10:515-9) used whole mount human atherosclerotic plaques to study differences in gene expression between fatty streaks and advanced lesions. They however, did not validate their findings on a large panel of individual patients and did not study the localization of differentially expressed genes.

The present invention relates to the use of a polynucleotide differentially expressed in ruptured (unstable) and stable atherosclerotic plaques as a marker for atherosclerosis wherein the polynucleotide is encoding an amino acid sequence selected from SEQ ID NO:1, SEQ ID NO:3 or SEQ ID NO: 5. It has now been found that said polynucleotides are either upregulated or downregulated in unstable atherosclerotic plaques.

Polynucleotides comprising SEQ ID NO:3 and SEQ ID NO:5 are already known in the art (e.g. from WO 9946380 and Accession Number AK 000362, respectively) as membrane spanning protein and human sorting nexin, respectively. There is no indication in the art, that these genes might be upregulated or downregulated in unstable plaque tissue.

A particular preferred embodiment of the invention relates to a novel polynucleotide that is highly expressed in unstable, ruptured atherosclerotic lesions. More specifically, the present invention provides for an isolated polynucleotide encoding the amino acid sequence SEQ ID:1. The term isolated denotes that the polynucleotide has been removed from its natural environment and is thus in a form suitable for use within genetically engineered protein production systems.

The invention also includes a polynucleotide comprising the DNA sequence which is indicated in SEQ ID NO: 2. In particular preferred is a polynucleotide comprising the complete coding DNA sequence of the nucleotides 1169-2587 of SEQ ID NO:2. Furthermore, to accommodate codon variability, the invention also includes sequences coding for the same amino acid sequences as the sequences disclosed herein (SEQ ID NO:1). Also portions of the coding sequences coding for individual domains of the expressed protein are part of the invention as well as allelic and species variations thereof Sometimes, a gene is expressed in a certain tissue as a splicing variant, resulting in the inclusion of an additional exon sequence, or the exclusion of an exon. Also a partial exon sequence may be included or excluded. A gene may also be transcribed from alternative promotors that are located at different positions within a gene, resulting in transcripts with different 5′ ends. Transcription may also terminate at different sites, resulting in different 3′ ends of the transcript. These sequences as well as the proteins encoded by these sequences all are expected to perform the same or similar functions and form also part of the invention.

The sequence information as provided herein should not be so narrowly construed as to require inclusion of erroneously identified bases. The specific sequence disclosed herein can be readily used to isolate the complete genes which in turn can easily be subjected to further sequence analyses thereby identifying sequencing errors.

The polynucleotides of this invention, which are differentially expressed in ruptured and stable atherosclerotic plaques, or the proteins encoded, are important tools for diagnostics and therapeutics.

As a diagnostic tool a differentially expressed gene can be used as a marker for unstable plaques in an individual, where the expression levels of the gene in tissue samples are determined. Further, it may be used to identify other sites of plaque instability in a patient that shows clinical symptoms of an unstable plaque of an artery, like the iliac artery, leading to peripheral ischernia. Since the long term prognosis of those patients is not determined by the success rate of the peripheral interventions, but by the occurrence of a myocardial or cerebral infarction, the correct diagnosis of all sites of plaque instability is of utmost importance. Diagnostic techniques such as imaging techniques, e.g. scintigraphy, may be applied, in which the radiolabeled unstable plaque specific gene is used as the target. Alternatively, the polynucleotides of this invention representing an unstable plaque specific gene, or the proteins encoded, may be used as serum/plasma markers, which may also be used to screen patients at risk for plaque instability or to evaluate the effects of other treatments. Further, the (novel) unstable plaque specific polynucleotides of this invention, or the encoded proteins or antibodies against the proteins, may be used to target other therapeutics to an unstable plaque.

Therefore, another aspect of the present invention is a method for determining the presence of a polynucleotide in a sample comprising: obtaining polynucleotides from an individual (e.g. by taking tissue samples, blood samples and the like, using (clinical) methods well known in the art for such purpose) and determining whether a polynucleotide encoding an amino acid sequence selected from SEQ ID NO:1, SEQ ID NO:3 or SEQ ID NO: 5 is present. Any method for detection of (poly)nucleotides known in the art for such purpose is included herewith. For example, nucleotide elongation methods/amplification methods may be considered, but also, such method may comprise the steps of: hybridizing to a sample a probe specific for a polynucleotide encoding an amino acid sequence selected from SEQ ID NO:1, SEQ ID NO:3 or SEQ ID NO: 5 under conditions effective for said probe to hybridize specifically to said polynucleotide and determining the hybridization of said probe to polynucleotides in said sample. The term “specific” in this respect means that the majority of hybridization takes place with a polynucleotide of this invention. Preferably, said probe comprises at least 25 of the nucleotides of SEQ ID NO:2, SEQ ID NO:4 or SEQ ID NO:6. More preferred, the probe comprises 50, and in particular preferred more than 100, nucleotides of SEQ ID NO:2, SEQ ID NO:4 or SEQ ID NO:6. Most preferred, the probe consists of a polynucleotide of nucleotides selected from the nucleotides 1169 to 2587 of SEQ ID NO:2. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

A further aspect of the present invention is a method for detecting in a sample a protein with amino acid sequence selected from SEQ ID NO:1, SEQ ID NO:3 or SEQ ID NO: 5, said method comprising: incubating with a sample a reagent that binds specifically to said protein (e.g. an antibody) under conditions effective for specific binding and determining the binding of said reagent to said protein in said sample.

In addition, a diagnostic process is an embodiment of the present invention comprising: determining the difference in expression level of a polynucleotide encoding an amino acid sequence selected from SEQ ID NO:1, SEQ ID NO:3 or SEQ ID NO: 5 in a sample derived from a host when compared to a known standard (e.g. using the above mentioned methods). Said known standard relates to healthy tissues and stable plaque material of healthy individuals. When the expression level of said polynucleotide of the present invention is upregulated or downregulated when compared to that standard, the host (usually a human being) from which the sample was derived, is at risk for atherosclerosis. Further, another aspect of this invention is a diagnostic process comprising: analyzing for the presence of the protein with amino acid sequence selected from SEQ ID NO:1, SEQ ID NO:3 or SEQ ID NO: 5 in a sample derived from a host (the methods for which analysis are well known in the art e.g. using the above mentioned methods). When the amount of the protein is higher or lower than the amount present in healthy tissue and/or stable plaque material, the host (usually a human being) from which the sample was derived, is at risk for atherosclerosis.

As a therapeutic tool, modulation—either directly or indirectly—of the expression of a polynucleotide encoding an amino acid sequence selected from SEQ ID NO:1, SEQ ID NO:3 or SEQ ID NO: 5, can increase plaque stability and thus inhibit the progression of atherosclerotic cardiovascular disease. For example, blocking antibodies and/or antagonists against an unstable plaque specific gene may be used to prevent the transition of a stable to an unstable plaque or to reverse an unstable plaque into a stable plaque. Further, the regulation of expression of the corresponding protein and the amount of the protein present in bodily tissues and fluids may be affected by regulation of the promotor of the gene or by the use of specifically synthesized antisense RNA for gene therapy.

The DNA according to the invention may be obtained from cDNA using suitable probes derived from SEQ ID NO:2, SEQ ID NO:4 or SEQ ID NO:6. Alternatively, the coding sequence might be genomic DNA, or prepared using DNA synthesis techniques. The polynucleotide may also be in the form of RNA. If the polynucleotide is DNA, it may be in single stranded or double stranded form. The single strand might be the coding strand or the non-coding (anti-sense) strand.

The present invention further relates to polynucleotides which have at least 70%, preferably 80%, more preferably 90%, even more preferred 95%, and highly preferably 98% and most preferred at least 99% identity with the entire DNA sequence of the nucleotides 1169-2587 of SEQ ID NO:2. Such polynucleotides encode polypeptides which retain the same biological function or activity as the natural, mature protein. Alternatively, also fragments of the above mentioned polynucleotides which code for domains of the protein which still are capable of binding to substrates are embodied in the invention.

The percentage of identity between two sequences can be determined with programs such as DNAMAN (Lynnon Biosoft, version 3.2). Using this program two sequences can be aligned using the optimal alignment algorithm of Smith and Waterman (1981, J. Mol. Biol, 147:195-197). After alignment of the two sequences the percentage identity can be calculated by dividing the number of identical nucleotides between the two sequences by the length of the aligned sequences minus the length of all gaps.

The present invention further relates to (the use of) polynucleotides having slight variations or having polymorphic sites. Polynucleotides having slight variations encode polypeptides which retain the same biological function or activity as the natural, mature protein.

The sequence of the newly identified polynucleotide of the present invention, SEQ ID NO:2, and the sequences SEQ ID NO:4 or SEQ ID NO:6 may also be used in the preparation of vector molecules for the expression of the encoded protein in suitable host cells. A wide variety of host cell and cloning vehicle combinations may be usefully employed in cloning the nucleic acid sequences coding for the proteins of SEQ ID NO:1, SEQ ID NO:3, SEQ ID NO:5 or parts thereof For example, useful cloning vehicles may include chromosomal, non-chromosomal and synthetic DNA sequences such as various known bacterial plasmids and wider host range plasmids and vectors derived from combinations of plasmids and phage or virus DNA.

Vehicles for use in expression of the polynucleotides of the present invention or a part thereof comprising a functional domain will further comprise control sequences operably linked to the nucleic acid sequence coding for the protein. Such control sequences generally comprise a promoter sequence and sequences which regulate and/or enhance expression levels. Of course control and other sequences can vary depending on the host cell selected.

Suitable expression vectors are for example bacterial or yeast plasmids, wide host range plasmids and vectors derived from combinations of plasmid and phage or virus DNA. Vectors derived from chromosomal DNA are also included. Furthermore an origin of replication and/or a dominant selection marker can be present in the vector according to the invention. The vectors according to the invention are suitable for transforming a host cell. Recombinant expression vectors comprising DNA of the invention as well as cells transformed with said DNA or said expression vector also form part of the present invention. Suitable host cells according to the invention are bacterial host cells, yeast and other fungi, insect, plant or animal host cells such as Chinese Hamster Ovary cells or monkey cells or human cell lines. Thus, a host cell which comprises DNA or expression vector according to the invention is also within the scope of the invention. The engineered host cells can be cultured in conventional nutrient media which can be modified e.g. for appropriate selection, amplification or induction of transcription. The culture conditions such as temperature, pH, nutrients etc. are well known to those ordinary skilled in the art.

The techniques for the preparation of DNA or the vector according to the invention as well as the transformation or transfection of a host cell with said DNA or vector are standard and well known in the art, see for instance Sambrook et al., Molecular Cloning: A laboratory Manual. 2nd Ed., Cold Spring Harbor Laboratory, Cold Spring Harbor, N.Y., 1989.

In another aspect of the invention, there is provided for a protein comprising the amino acid sequence encoded by the above described DNA molecules. Preferably, the protein according to the invention comprises an amino acid sequence shown in SEQ ID NO:1. Also part of the invention are proteins resulting from post translational processing, which proteins are encoded by the polynucleotide of this invention.

Also functional equivalents, that is proteins homologous to amino acid sequences SEQ ID NO:1, SEQ ID NO:3, SEQ ID NO:5 or parts thereof having variations of the sequence while still maintaining functional characteristics, are included in the invention.

The variations that can occur in a sequence may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities, have been described. Amino acid replacements between related amino acids or replacements which have occurred frequently in evolution are, inter alia Ser/Ala, Ser/Gly, Asp/Gly, Asp/Asn, Ile/Val (see Dayhof M. D., Atlas of protein sequence and structure, Nat. Biomed. Res. Found., Washington D.C., 1978, vol. 5, suppl. 3). Based on this information Lipman and Pearson developed a method for rapid and sensitive protein comparison (Science, 1985, 227, 1435-1441) and determining the functional similarity between homologous polypeptides. It will be clear that also polynucleotides coding for such variants are part of the invention.

The polypeptides according to the present invention also include polypeptides comprising SEQ ID NO:1, but further polypeptides with a identity of at least 70%, preferably 80%, more preferably 90%, and even more preferred 95%. Also portions of such polypeptides still capable of conferring biological effects are included. Especially portions which still bind to targets form part of the invention. Such portions may be functional per se, e.g. in solubilized form or they might be linked to other polypeptides, either by known biotechnological ways or by chemical synthesis, to obtain chimeric proteins. Such proteins might be useful as therapeutic agent.

The proteins according to the invention can be recovered and purified from recombinant cell cultures by common biochemical purification methods (as decribed in Havelaar et al, J. Biol. Chem. 273, 34568-34574 (1998)) including ammonium sulfate precipitation, extraction, chromatography such as hydrophobic interaction chromatography, cation or anion exchange chromatography or affinity chromatography and high performance liquid chromatography. If necessary, also protein refolding steps can be included. Alternatively the protein can be expressed and purified as a fusion protein containing (“tags”) which can be used for affinity purification.

The proteins according to the present invention may be used for the in vitro or in vivo identification of novel targets or analogues. For this purpose e.g. binding studies may be performed with cells transformed with DNA according to the invention or an expression vector comprising DNA according to the invention, said cells expressing an unstable plaque specific polynucleotide according to the invention.

Alternatively also the (newly identified) polynucleotides according to the invention as well as the target-binding domain thereof may be used in an assay for the identification of functional targets or analogues for the gene.

Using such an assay compounds can be identified that prevent the transition of a stable to an unstable plaque or reverse that prevent the transition of a stable to an unstable plaque or reverse an unstable plaque into a stable plaque an unstable plaque into a stable plaque, and thus inhibit the progression of atherosclerotic disease.

Thus, the present invention provides for a method for identifying compounds that prevent the transition of a stable to an unstable plaque or reverse an unstable plaque into a stable plaque. The method comprises the steps of

a) introducing into a suitable host cell a nucleotide encoding the amino acid selected from the sequences of SEQ ID NO:1, SEQ ID NO:3 and SEQ ID NO:5;

b) culturing the host cells under conditions to allow expression of the introduced DNA sequence;

c) bringing the host cell of step b, or products thereof, into contact with compounds potentially effecting the function of the expressed protein with SEQ ID NO:1, SEQ ID NO:3 or SEQ ID NO: 5;

d) determining the effect of such compounds on the function of the expressed protein.

The present invention thus provides for a quick and economic method to screen for therapeutic agents for the prevention and/or treatment of cardiovascular diseases related to the transition of a stable to an unstable plaque.

The invention also provides for a method for the formulation of a pharmaceutical composition comprising mixing modulator compounds identified according to the above procedure with a pharmaceutically acceptable carrier.

Pharmaceutical acceptable carriers are well known to those skilled in the art and include, for example, sterile saline, lactose, sucrose, calcium phosphate, gelatin, dextrin, agar, pectin, peanut oil, olive oil, sesame oil and water.

Furthermore the pharmaceutical composition according to the invention may comprise one or more stabilizers such as, for example, carbohydrates including sorbitol, mannitol, starch, sucrosedextrin and glucose, proteins such as albumin or casein, and buffers like alkaline phosphates. Methods for making preparations and intravenous admixtures are disclosed in Remingtons's Pharmaceutical Sciences, pp. 1463-1497 (16th ed. 1980, Mack Publ. Co of Easton, Pa., USA).

Thus, the modulator compounds identified by using a polynucleotide according to the invention are useful in the preparation of a pharmaceutical. The pharmaceutical is to be used in atherosclerotic disorders.

Also within the scope of the invention are antibodies, especially monoclonal antibodies raised against a protein according to the invention. Such antibodies may be used both therapeutically and diagnostically. The antibodies can be prepared according to methods known in the art e.g. as described in EP488470.

The invention is further explained by reference to the following illustrative Examples.

### LEGEND TO THE FIGURES

FIG. 1. Inverse Northern Dot Blot analysis of cDNA clones generated by SSH. Two identical Dot Blots were made by transfer of PCR products to nylon membranes. The Dot Blots were hybridized to A) a ³²P-labeled cDNA pool of 3 different stable plaques and B) a ³²P-labeled cDNA pool of 3 different ruptured plaques as described in the Examples. The position of SEQ ID NO:2, a clone upregulated in ruptured plaques, is D6.

FIG. 2. RT-PCR analysis of the newly identified polynucleotide of SEQ ID NO:2, differentially expressed in ruptured or stable human atherosclerotic plaques. The figure shows expression in 10 different stable plaques (left panel) and 10 different ruptured plaques (right panel).

FIG. 3. Inverse Northern Dot Blot analysis of cDNA clones generated by SSH. Two identical Dot Blots were made by transfer of PCR products to nylon membranes. The Dot Blots were hybridized to A) a ³²P-labeled cDNA pool of 3 different stable plaques and B) a ³²P-labeled cDNA pool of 3 different ruptured plaques as described in the Examples. The position of SEQ ID NO:6, a clone downregulated in ruptured plaques, is C3.

FIG. 4. Expression of SSH6 in the vascular wall. RT-PCR on mRNA isolated from veins and arteries with atherosclerotic lesions in various stages.

FIG. 5. Tissue distribution of the ubiqutiously expressed SSH 6 mRNA. Hybridization of a human multiple tissue array with the ³³P-dCTP labeled cDNA seq of the nucleotides 905-1341 of SEQ ID NO:2. A schematical representation of the various tissues and cell lines is depicted in the lower panel.

FIG. 6. Tissue distribution of SSH6v mRNA. Hybridization of the human multiple tissue array with 33 P-dCTP labeled exon 3 (see schematical representation in the lower panel of FIG. 5 for blot composition). The lower panel shows hybridization with VSMC derived RNA and exon 3 containing plasmid DNA as a positive control.

FIG. 7. Schematic representation of the GST-SSH6 fusion protein. The 302 C-terminal AA deduced from the putative open reading of SSH6 are fused to the C-terminal end of glutathion S-transferase (GST).

FIG. 8. SSH6 protein expression. Western blot analysis of human atherosclerotic plaques, human plasma and several human tissue lysates and cell lines using the SSH6 specific SSH6-scFv. Lane 1: smooth muscle cell lysate, lane 2: human aorta, lane 3: LS174T cells, lane 4: LLC cells, lane 5: CaCo cells, lane 6: COS cells, lane 7: marker, lane 8: ruptured atherosclerotic plaque, lane 9: HUVEC cells, lane 10: OVCAR cells, lane 11: human plasma.

### EXAMPLES

General

The technique of suppression subtractive hybridization (SSH) (Diatchenko L, et al., Proc Natl Acad Sci USA 1996; 93:6025-30) was applied, using whole mount specimens, for the identification of the polynucleotide of the present invention which is differentially expressed in whole mount human stable and ruptured plaques.

To obtain plaque type specific genes, the plaques included in the 2 pools (ruptured and stable plaques) were morphologically diverse with respect to the presence of a lipid core, calcium deposition and the amounts of inflamnnatory cells. Furthermore, the SSH procedure was performed on pools of 3 advanced stable lesions (type IV and V) and 3 ruptured lesions (type VI), to circumvent patient based differences. To select for genes with larger differences in expression the SSH was performed with a 4-fold excess of driver. The SSH procedure yielded a cDNA library, enriched with clones upregulated in ruptured plaques. Differential expression of a number of randomly chosen clones was validated by Inverse Northern Dot Blot (INDB) analysis. Sequences showing an at least 2-fold difference in expression were sequenced. To validate the reproducibility of expression of these sequences, RT-PCR analysis was performed on a larger series of individual ruptured (n=10) and individual stable (n=10) plaques, which showed a striking consistency of expression for the polynucleotide of the present invention, present in 8 ruptured and 2 stable plaques.

The differential expression pattern of the polynucleotide of the present invention suggests a potential role for this gene in plaque rupture.

Tissue Sampling and RNA Isolation

Plaques were obtained from patients undergoing vascular surgery (Department of General Surgery, Academic Hospital Maastricht). Patient characteristics are summarized in Table 1. Immediately after resection, the atherosclerotic specimen was divided into parallel parts of 5 mm for RNA isolation and histological analysis. Tissue destined for RNA isolation was immediately frozen in liquid nitrogen and stored at −80° C. Total RNA was isolated using the guanidine isothiocyanate/CsCl method (Chomczynski P., et al., Anal Biochem 1987;162:156-9). Specimens for histological analysis were fixed in 10% phosphate buffered formalin (pH 7.4), routinely processed and embedded in paraffin. Sections were cut, stained with heamatoxylin and eosin and classified according to the morphological criteria of the American Heart Association (Stary H. C., et al., Arterioscler Thromb Vasc Biol 1995;15:1512-31). Only advanced atherosclerotic plaques were included in the study. Type IV and V lesions were defined as stable plaques and type VI lesions were defined as ruptured plaques.

### Example 1

Suppression Subtractive Hybridization

The SSH procedure was performed using the PCR-Select cDNA Subtraction Kit (Clontech) essentially according to the protocol of the manufacturer, with minor adjustments. Briefly, total RNA was isolated from whole mount plaques of 6, age matched, male patients undergoing peripheral vascular surgery (Table 1). To correct for patient based differences in gene expression, 2 pools of total RNA were generated. Pool 1 contained 1 μg of total RNA derived from 3 ruptured plaques of 3 individual patients. Pool 2 contained 1 μg of total RNA derived from 3 stable plaques of 3 individual patients. The SMART™ PCR cDNA Synthesis Kit (Clontech) was used for the preparation and amplification of double stranded cDNA. In the forward reaction, genes upregulated in ruptured plaques were isolated. RsaI digested tester cDNA was ligated to two different adaptors and hybridized to a 4-fold excess of driver cDNA to enrich for differentially expressed genes. Differentially expressed genes were amplified by 2 rounds of PCR. The resulting fragments were gel purified, cloned into the pGEMT-easy vector (Promega) and subsequently transformed to highly competent E. coli JM109 cells (Promega). The thus constructed (forward subtracted) library contained a number of clones upregulated in ruptured atherosclerotic plaques.

### Example 2

Analysis of Subtracted cDNA Libraries

Inverse Northern Dot Blotting (INDB)

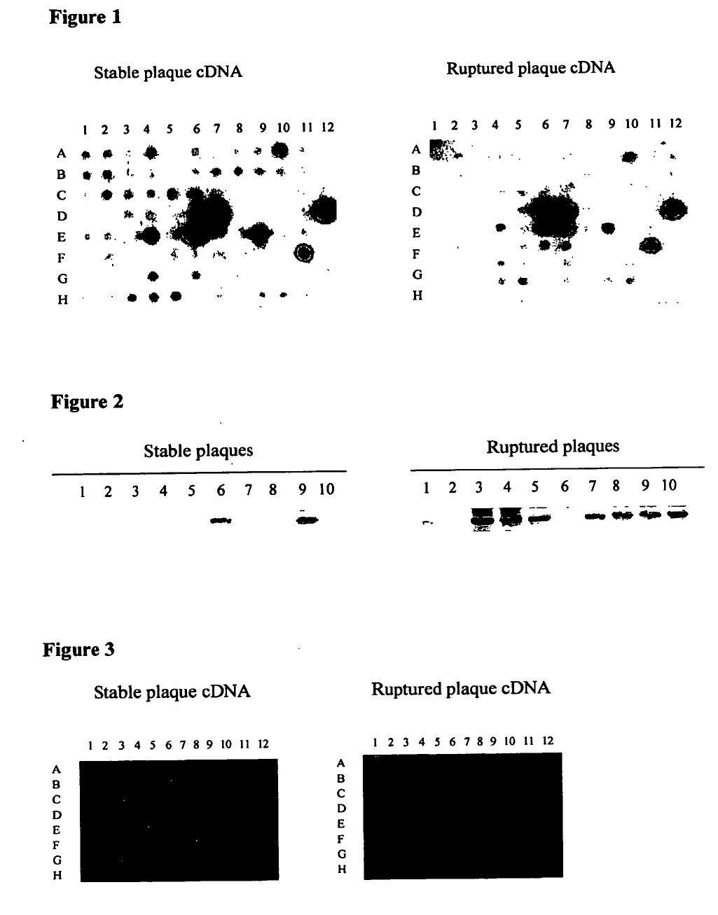

Differential expression of the sequences of the SSH library was verified by a second independent method, the INDB analysis. Sequences of the library were randomly chosen and screened for expression in ruptured and stable plaques. This resulted in the identification of a clone that was uniquely expressed in ruptured plaques (FIG. 1).

Procedure: Inserts were amplified by PCR using the T7 (5′-TAATACGACTCACTATAGGG-3′, SEQ ID NO:7) and SP6 (5′-ATTTAGGTGACACTATA-3′, SEQ ID NO: 8) primers under standard conditions. Briefly, 10 μl of PCR product was diluted in 200 μg 6×SSC, heated to 95° C. and quenched on ice. Two identical dot blots were made by transferring 100 μl of the sample to a nylon membrane (Nytran; Schleicher & Schuell) using a 96-wells BioRad Dot Blot apparatus and the DNA was subsequently crosslinked by UV irradiation. The filters were hybridized at high stringency with ³²P-labeled (High Prime, Boehringer Mannheim) SMART™ cDNA of either stable or ruptured plaques using standard procedures. Hybridization signals were normalized using RNA-polymerase II and genomic DNA signals. Quantitative analysis was performed by phosphor image analysis.

### Example 3

Sequencing

The differentially expressed polynucleotide of Example 2 was sequenced using the Thermo Sequenase fluorescent labelled primer (M13 reverse 5′-TTTCACACAGGAAACAGGAAACAGCTATGAC-3′, SEQ ID NO: 9, M13 forward 5′-CGCCAGGGTTTTCCCAGTCAC GAC-3′, SEQ ID NO: 10) cycle sequencing kit (Amersham Pharmacia Biotech) and analyzed on an ALF-express automatic sequencer.

The cDNA clone contained an insert of 540 base pairs, of which 344 nucleotides were sequenced.

A search for sequences homologous or identical to these 344 nucleotides in a gene database from INCYTE revealed a template of 2098 nucleotides, containing an open reading frame in the 3′ part of the sequence, but lacking a stop codon. This template was used to search for overlapping sequences. The templates found in this way were assembled and hand-edited to reveal a sequence of 3835 nucleotides (SEQ ID NO:2) with an open reading frame of 473 amino acids coding for a protein with a calculated molecular weight of 53.3 kDa (SEQ ID NO:1).

### Example 4

RT-PCR

To further validate the expression profile found in the INDB, RT-PCR analysis on 10 ruptured and 10 stable plaques was performed. To exclude patient- and artery-biased expression, plaques originated from several arteries of different patients (Table 1). Expression was normalized to the expression level of GAPDH, which expression level was comparable between different samples (FIG. 2).

Procedure: Isolation of total RNA was carried out as described above. The SMART™ PCR cDNA Synthesis Kit (Clontech) was used for the preparation of double stranded cDNA from 0.5 μg template RNA. cDNA was diluted to a total volume of 50 μl. PCR amplification of the polynucleotide (sense: 5′-GGCTAATTCGGGAGATAGCC-3′, SEQ ID NO: 11, +antisense: 5′-CAACACCTCATGGCAAGTCC-3′, SEQ ID NO: 12) was performed on 1 μl of first strand cDNA using standard conditions (30 cycles of denaturation for 1 min at 94° C., annealing for 1 min at 55° C. and extension for 1 min at 72° C.). Resulting PCR products of approximately 300 bp were analyzed on a 1% agarose gel.

Result: Expression of the polynucleotide of SEQ ID NO: 2 was found in 8 out of 10 ruptured plaques, while only 2 out of 10 stable plaques tested positive.

TABLE 1
Patient characteristics

| plaque type | No | sex* | age | Artery | used for |
|---|---|---|---|---|---|
| Ruptured | 1 | m | 60 | Abdominal aorta | RT-PCR^{†} |
| | 2 | f | 66 | Common femoral | RT-PCR |
| | | | | artery | |
| | 3 | m | 72 | Abdominal aorta | SSH^{‡}/INDB^{§}/RT-PCR |
| | 4 | m | 74 | Abdominal aorta | RT-PCR |
| | 5 | m | 73 | Abdominal aorta | SSH/INDB/RT-PCR |
| | 6 | m | 55 | Femoral artery | RT-PCR |
| | 7 | m | 75 | Abdominal aorta | SSH/INDB/RT-PCR |
| | 8 | m | 73 | Femoral artery | RT-PCR |
| | 9 | m | 63 | Abdominal aorta | RT-PCR |
| | 10 | m | 58 | Carotid artery | RT-PCR |
| Stable | 11 | m | 72 | Carotid artery | RT-PCR |
| | 12 | f | 67 | Carotid artery | RT-PCR |
| | 13 | m | 57 | Carotid artery | RT-PCR |
| | 14 | f | 71 | Femoral artery | RT-PCR |
| | 15 | m | 78 | Common femoral | SSH/INDB/RT-PCR |
| | | | | artery | |
| | 16 | m | 78 | Common iliac | SSH/INDB/RT-PCR |
| | | | | artery | |
| | 17 | m | 60 | Abdominal aorta | RT-PCR |
| | 18 | m | 68 | Carotid artery | RT-PCR |
| | 19 | m | 67 | Carotid artery | SSH/INDB/RT-PCR |
| | 20 | m | 70 | Carotid artery | RT-PCR |

*f: female
m: male
^{†}RT-PCR: reverse transcriptase PCR
^{§}INDB: inverse northern dot blot
^{‡}SSH: suppression subtractive hybridization

### Example 5

SSH6: A Vasular Smooth Muscle Cell Specific mRNA and Protein Plaque rupture of atherosclerotic plaques is the predominant underlying process in the pathogenesis of acute coronary syndromes and peripheral vascular disease. Insight into the pathways that destabilize plaques is sparse. Suppressive Subtractive Hybridization (SSH) analysis on human atherosclerotic plaques-derived RNA, resulted in the identification of a large library of cDNAs differentially expressed in stable and ruptured atherosclerotic plaques (see Example 1). Differential expression of the sequences of the SSH library was verified by inverse northern dot blot (INDB) or macro-array analysis (see Example 2). One of these cDNA clones, SSH 6, was over 2 fold upregulated in ruptured plaques. This clone contained a cDNA insert of 436 bp (the nucleotides 905-1341 of SEQ ID NO:2), containing a putative ORF of 57 amino acids (amino acids 1-57 of SEQ ID NO:1).

To further validate the expression profile found in INDB, RT-PCR analysis on 10 ruptured and 10 stable plaques was performed. To exclude patient- and artery-biased expression, plaques originated from several arteries of individual patients (see Table 1, Example 4). Expression was normalized to the expression level of GAPDH, which was comparable between different samples. Expression of this sequence was found in 8 out of 10 ruptured plaques, while only 2 out of 10 stable plaques tested positive. (see Example 4)

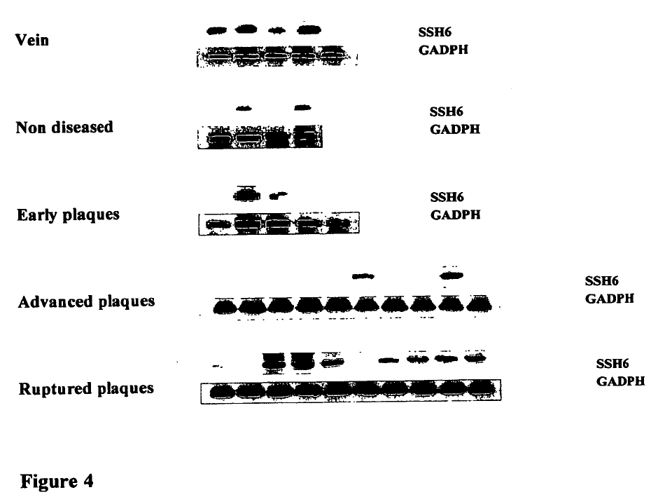

RT-PCR on individual samples of veins (n=5), non-diseased artery (n=4) and early atherosclerotic plaques (n=5) revealed SSH6 expression in all veins, in 50% of non-diseased arteries, and in 40% of early lesions, respectively (FIG. 4).

A search for sequences homologous or identical to the SSH 6 sequence revealed several templates, including a template of 2098 nucleotides in a INCYTE gene database, showing partial overlap. However, clone SSH 6 contained an insert of 120 nt (the nucleotides 1112-1231 of SEQ ID NO: 2) in comparison to the majority of sequences in the databases. This 120 nt insert contains a putative start codon (the nucleotides 1169-1171 of SEQ ID NO: 2) in frame with a large ORF. In order to obtain a full length SSH 6 clone, a Vascular Smooth Muscle Derived (VSMC) derived cDNA library was screened with the original cDNA fragment (kindly provided by Dr C A de Vries, AMC, Amnsterdam). This screening resulted in the isolation of numerous (>10) cDNA clones, all containing over 2000 bp of SSH6 sequences. Sequence analysis of the largest clone revealed the presence of a 2858 nt cDNA fragment (identical to the nucleotides 64-2920 of SEQ ID: NO 2, with the exception that in this fragment an additional “g” nucleotide is present between nucleotides 2904 and 2905 of SEQ ID NO:2, which is in the non-coding part of the sequence), containing an ORF of 473 amino acids (SEQ ID: NO 1).

Detailed bio-infornatics using public domain and INCYTE databases indicated the presence of a putative “vascular wall specific” mRNA and vascular wall specific protein, further indicated as SSH6v. Aligmnent of SSH6v cDNA and genomic databases showed that the SSH6 gene is spanning a genomic region of over 90 Kb on chromosome 5 p13 and consists of at least 12 exons (see Table 2).

TABLE 2
Genomic organization of SSH6

| | Position (from | | | |
|---|---|---|---|---|
| | nucleotide . . . | | | |
| | to . . . in | | | |
| exon | SEQ ID NO: 2) | intron | length | |
| 1 | 1-158 | | | |
| | | 1 | 1236 | bp |
| 2 | 159-1111 | | | |
| | | 2 | >37 | Kb |
| 3 | 1112-1231 | | | |
| | | 3 | >16 | Kb |
| 4 | 1232-1356 | | | |
| | | 4 | 128 | bp |
| 5 | 1357-1579 | | | |
| | | 5 | 4644 | bp |
| 6 | 1580-1646 | | | |
| | | 6 | >10 | Kb |
| 7 | 1647-1831 | | | |
| | | 7 | 590 | bp |
| 8 | 1832-1972 | | | |
| | | 8 | >10 | Kb |
| 9 | 1973-2140 | | | |
| | | 9 | >1.5 | Kb |
| 10 | 2141-2328 | | | |
| | | 10 | >10 | Kb |
| 11 | 2329-2431 | | | |
| | | 11 | 1311 | |
| 12 | 2432->2920 | | | |

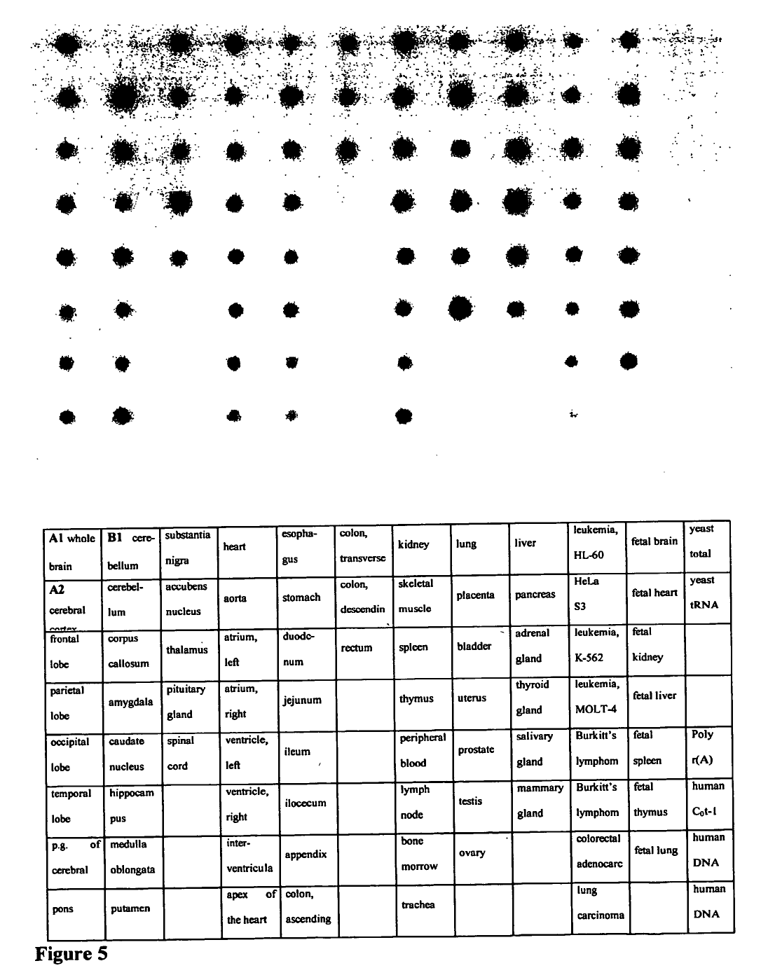

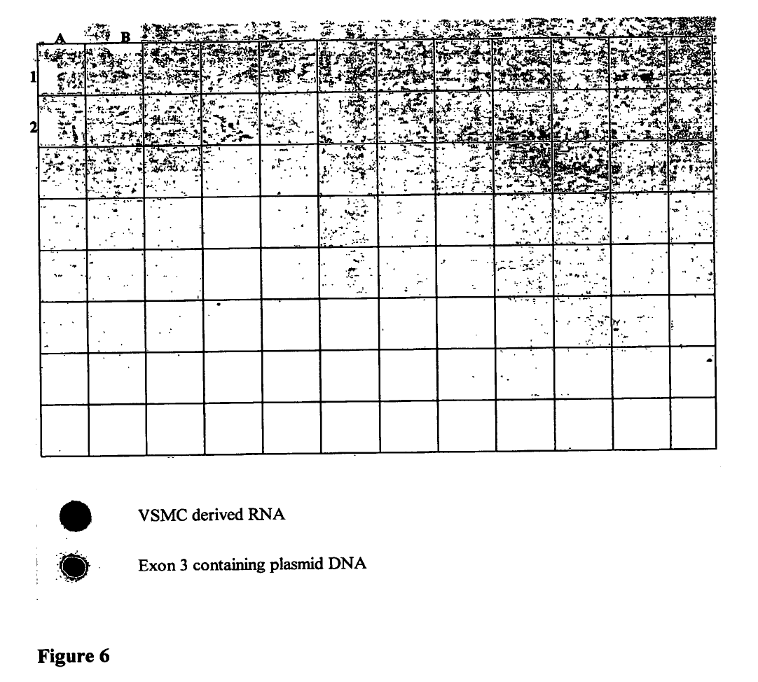

The vascular wall specific mRNA/protein SSH6v and the ubiquitously expressed SSH6 mRNA result from alternative splicing of exon 3 (nucleotides 1112-1231 of SEQ ID NO: 2). The differential issue distribution of SSH6v and SSH6 was further substantiated by multi-tissue northern blot analysis on 62 adult human tissues, 8 human cell lines, 7 fetal tissues and 6 controls (Multiple Tissue Expression Array MTE: Clontech, Palo Alto, Calif., USA). FIG. 5 shows the tissue distribution of SSH 6 (using the 436 bp cDNA insert—the nucleotides 905-1341—of SEQ ID NO: 2 as a probe), while FIG. 6 indicates the vascular wall specific expression of the SSH6v messenger (using a 120 nt exon-3 specific probe). The bottom panel of FIG. 6 indicates hybridization of this probe to VSMC derived RNA and a positive control (full length SSH6v cDNA).

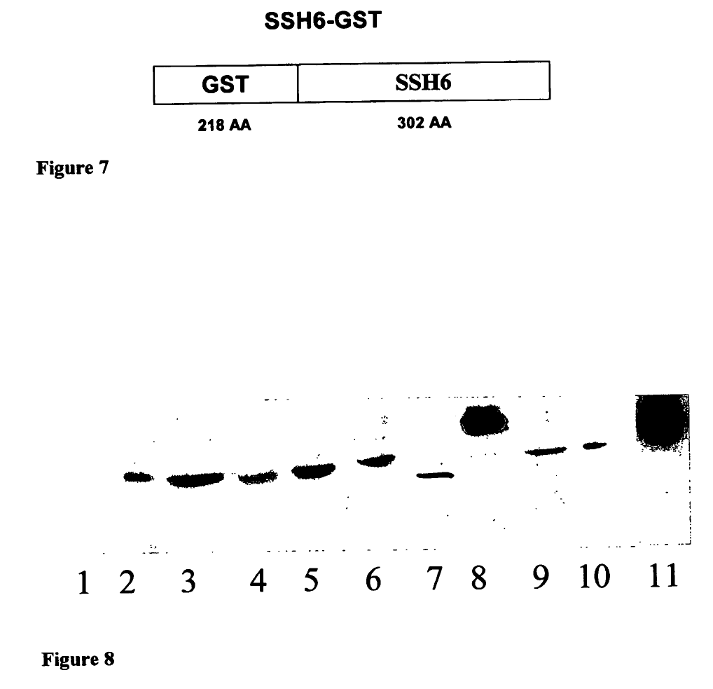

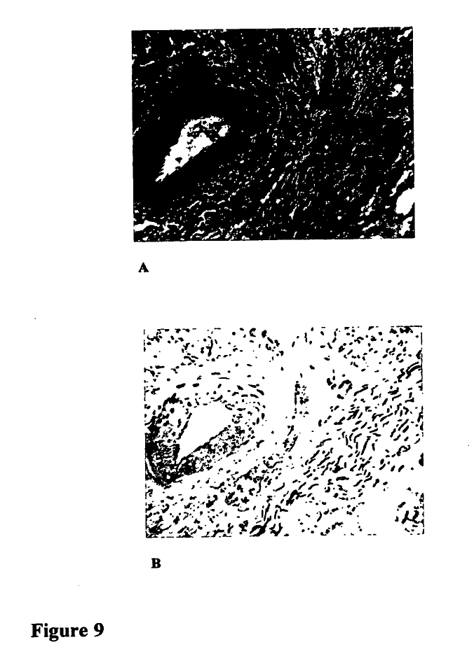

In order to develop immunological tools to characterize the SSH6 protein in more detail, part of the ORF (909 bp) was fused to glutathione S-transferase (67 kDa) and the resulting recombinant protein was used to select SSH6-specific single-chain Fv fragments (scFv) (see FIG. 7 for a schematical representation). Western blot analysis of human atherosclerotic plaques, human plasma, human tissue lysate and several cell lines using a SSH6 specific scFv revealed the presence of a protein of the expected size (˜53 kDa) in vascluar wall derived lysates and plasma only, and a 35 kDa product in the majority of lysates (see FIG. 8). This 35 kDa protein most likely is the result of translation start at an internal Methionine (AA 172) Furthermore, immunohistochemical analysis of human ruptured atherosclerotic plaques indicated SSH6 protein expression in vascular smooth muscle cells (VSMC) (see FIG. 9). Interestingly, localization of the SSH6 protein nicely correlates to the observed presence of SSH6 mRNA in primary cultures of human VSMC derived from a ruptured plaque.

Conclusion: a previously unknown vascular wall specific mRNA/protein SSH6v has been identified that is expressed in human VSMC and is upregulated in ruptured atherosclerotic plaques.

### Materials and Methods Example 5

RT-PCR Analysis

To reveal the expression profile of SSH6v in the vascular wall, RT-PCR was performed on mRNA isolated from veins and arteries with atherosclerotic lesions in various stages and on mRNA isolated from a primary culture of VSMC derived from ruptured atherosclerotic lesions. RNA isolation, cDNA synthesis and RT-PCR was performed as described previously. In brief, a SSH6v-specific DNA fragment of 217 bp was amplified by PCR on first strand cDNA using the sense primer (5′-GGCTAATTCGGGAGATAGCC-3′, SEQ ID NO: 11) and antisense primer (5′-CAACACCTCATGGCAAGTCC-3′, SEQ ID NO: 12) under standard conditions (30×(94° C., 1 min; 55° C., 1 min; 72° C., 1 min). The resulting PCR products were analyzed on a 1% agarose gel.

Multi-tissue Northern Blot Analysis

Multi-tissue northern blot was performed using the Multiple Tissue Expression Array MTE (Clontech, Palo Alto, Calif., USA) essentially according the protocol of the manufacturer. Briefly, the MTE array was hybridized with denatured ³³P-labeled cDNA probes for 12 hours at 65° C. and exposed to x-ray film at −70° C. during 12 hours.

Construction of the Glutathione S-Transferase-SSH6 Fusion Protein (GST-SSH6) Expression Plasmid

The C-terminal part of the SSH-6 cDNA was amplified using the sense primer 5′-CCTAAATCTAGAGCGTCGACGATGCTGG-3′ (SEQ ID NO: 13) and antisense primer 5′-AAGCTGTTAGTCGACCCTTCACA-3′ (SEQ ID NO: 14) in order to introduce a SalI restriction site for the construction of the expression plasmid. Simultaneously with the introduction of the desired restriction sites, a proline (CCA) and arginine (AGG) codon inside the open reading frame of SSH6 were mutated into a serine (TCG) and threonine (ACG) codon, respectively. Subsequently, the PCR product was digested with Sal I and the resulting 938 bp fragment was ligated in pGEX-4T-2 and transformed to BL21 E. coli cells. In order to produce GST protein BL21 E. coli cells were transformed with pGEX4T-2 without additional insert.

Western Blot Analysis of SSH6

Lysates of various human tissues and cell lines were prepared as follows: 2-5×10⁷ cells were collected, resuspended in 500 μl ice cold lysis buffer (25 mM Tris-HCl (pH 7.5), containing 150 mM NaCl, 1 mM EDTA, 2 mM PMSF, 1 mM DTT, 0.1 mM benzamidine and 1% Nonidet P40) and incubated for 20 min on ice. The cell lysates were cleared by centrifugation. Lysates equivalent to 10-20 μg of total proteins or serial dilutions of human plasma were separated by SDS-PAGE (9%) and transferred onto nitrocellulose. After blocking with PBS containing 2% (w/v) skimmed milk powder (MPBS), blots were stained for 1 h with 5 μg/ml of purified anti SSH6-scFv. Following incubation with HRP labeled anti-myc antibody HRP activity was visualized by ECL staining.

Immunohistochemical Analysis

Four μm frozen atherosclerotic plaques sections were pre-treated with TBS-TS (TBS, 0.1% (v/v) Tween 20, 3% human serum and 3% sheep serum). Subsequently, sections were incubated for 30 min with scFv-2A4. Bound scFv antibodies were detected with an anti myc-antibody (9E10), followed by incubation with biotinylated sheep anti-mouse antibody and an alkaline phosphatase coupled ABC reagent. Alkaline phosphatase activity was visualized using the Alkaline Phosphatase Kit I (Vector) containing 1 mM levamisole (Sigma), resulting in a red precipitate. The sections were counterstained with hematoxylin.

### Example 6

According to the procedures described in Examples 1-4, also SEQ ID NO:4 was identified, another clone upregulated (3-fold) in unstable plaques.

Specific sequencing:

The cDNA clone contained an insert of 1050 base pairs, of which 391 nucleotides were sequenced.

A search for sequences homologous or identical to these 391 nucleotides in the INCYTE gene database revealed a sequence of 3145 nucleotides (SEQ ID NO:4), containing an open reading frame of 946 amino acids (SEQ ID NO:3). This open reading frame corresponds to a protein with similarity to the human sorting nexin (GenBank accession number AK 000362).

### Example 7

According to the procedures described in Examples 1-4, also SEQ ID NO:6 was identified, a clone downregulated in unstable plaques (specific for stable plaques). In FIG. 3, a INDB analysis of the polynucleotide of SEQ ID NO:6 is shown.

Specific sequencing:

The cDNA clone contained an insert of 400 base pairs, of which 348 nucleotides were sequenced.

A search for sequences homologous or identical to these 348 nucleotides in the Geneseq patent database revealed a sequence of 4117 nucleotides (SEQ ID NO:6), containing an open reading frame of 950 amino acids (SEQ ID NO:5). This open reading frame has been predicted to encode a membrane spanning protein, MSP-5 (WO 9946380).

1. A use of a polynucleotide differentially expressed in ruptured and stable atherosclerotic plaques as a marker for atherosclerosis wherein the polynucleotide is encoding an amino acid sequence selected from SEQ ID NO:1, SEQ ID NO:3 or SEQ ID NO: 5.

2. A method for determining the presence of a polynucleotide in a sample comprising:
obtaining polynucleotides from an individual and determining whether a polynucleotide encoding an amino acid sequence selected from SEQ ID NO:1, SEQ ID NO:3 or SEQ ID NO: 5 is present.

3. The method according to claim 2 where the polynucleotide is a RNA polynucleotide.

4. The method according to claim 2 or 3 comprising the steps of:
hybridizing to a sample a probe specific for the polynucleotide encoding an amino acid sequence selected from SEQ ID NO: 1, SEQ ID NO: 3 or SEQ ID NO: 5 under conditions effective for said probe to hybridize specifically to said polynucleotide and
determining the hybridization of said probe to polynucleotides in said sample.

5. A method for detecting in a sample a protein with amino acid sequence selected from SEQ ID NO: 1, SEQ ID NO: 3 or SEQ ID NO: 5, said method comprising:
incubating with a sample a reagent that binds specifically to said protein under conditions effective for specific binding and
determining the binding of said reagent to said protein in said sample.

6. A diagnostic process comprising:
determining the difference in expression level of a polynucleotide encoding an amino acid sequence selected from SEQ ID NO: 1, SEQ ID NO: 3 or SEQ ID NO: 5 in a sample derived from a host when compared to a known standard.

7. A diagnostic process comprising:
analyzing for the presence of a protein with amino acid sequence selected from SEQ ID NO: 1, SEQ ID NO: 3 or SEQ ID NO: 5 in a sample derived from a host.

8. An isolated polynucleotide comprising a nucleic acid sequence encoding the amino acid sequence SEQ ID NO: 1.

9. The polynucleotide according to claim 8 comprising the nucleic acid sequence SEQ ID NO:2.

10. The polynucleotide according to claim 8 or 9 comprising the nucleotides 1169 to 2587 of SEQ ID NO: 2.

11. The polynucleotide according to any one of claims 8-10 consisting of a nucleic acid sequence encoding the amino acid sequence SEQ ID NO: 1.

12. The polynucleotide according to any one of claims 8-11 consisting of the nucleic acid sequence SEQ ID NO: 2. 3
